(19) United States
(12) Patent Application Publication
(10) Pub. No.: US 2015/0087049 A1
(43) Pub. Date: Mar. 26, 2015

(54) Research Photobioreactor

(71) Applicant: Celldeg GbR, Berlin (DE)

(72) Inventors: Rudolf Ehwald, Berlin (DE); Lars Bähr, Berlin (DE); Arne Wüstenberg, Berlin (DE)

(21) Appl. No.: 14/489,536

(22) Filed: Sep. 18, 2014

(30) Foreign Application Priority Data
Sep. 25, 2013 (DE) 102013015969.5

(51) Int. Cl. C12M 1/00; C12M 1/34

(52) U.S. Cl. CPC C12M 21/02; C12M 41/44; C12M 29/04
USPC 435/287.1; 435/292.1

(57) ABSTRACT

The invention relates to a research photobioreactor for growth at extremely high cell densities in axenic cultures of cyanobacteria and microalgae exposed to a high light intensity. A hydrophobic gas-permeable first membrane situated at the bottom of the reaction chamber serves for the entry of CO₂ into the cell suspension. The first membrane separates the CO₂ containing gaseous phase in a basis chamber from the cell suspension that covers the membrane. It has a permeability coefficient for the gases of air many times that of the physically feasible rate of absorption of these gases in water. The turbulent flow within the cell suspension is obtained by shaking. A high shearing rate hereby realized increases the CO₂ absorption rate in the liquid phase close to the membrane, thus avoiding C-limitation. A porous hydrophobic gas-permeable second membrane separates the reaction chamber from at least one gas exchange chamber.

### CROSS REFERENCE TO RELATED APPLICATIONS

This application claims the benefit of German Application No. DE 102013015969.5 filed on Sep. 25, 2013; the German priority application is incorporated by reference herein in its entirety.

### BACKGROUND

At low biomass concentrations cyanobacteria and micro-algae reveal high specific rates of the photoautotrophic growth (up to more than 3 d⁻¹) and exhibit a high potential for bioprocess techniques to produce biomass or valuable organic materials (foods or animal feeds, drugs, biofuels) by photosynthesis. Axenic research culture of photoautotrophic microorganisms (PMO) is carried out mainly in shaken culture vessels exposed to the light or in vessels wherein a flow of bubbles containing air enriched with CO₂ is rising up, while the entrance of foreign organisms is prevented by bacterial filters.

Photobioreactors for use in laboratories (research-photobioreactors) have been developed for standardization and optimization of the conditions controlling photoautotrophic growth. However, independent of the volume of the reaction vessels, it is difficult to ensure the provision of PMO with nutrients, CO₂ and light in such a manner that a high biomass concentration can be reached and intensive growth is possible at high biomass concentrations. A concentration of the dehydrated biomass (DB) above 5 μl⁻¹ has to be designated as extremely high at the culture of PMO (e.g. Wang et al. 2012). ‘Besides the maintenance of saturating concentrations of inorganic carbon or other miner nutrients in the culture medium it is required to supply light quanta to all cells and to avoid oxidative stress in order to obtain a high rate of photosynthesis referred to the volume of the cell suspension or to the area exposed to light. According to the present art, extremely high concentrations of dehydrated biomass (CDB) can be obtained in short time, when cell suspensions of relatively low thickness (below 2 cm) are put in turbulent flow at photon flux densities (PFD) above 1 mmol m⁻² s⁻¹. Even at extremely high PFD (several mmol m⁻² s⁻¹) at the exposed surface of a dense cell suspension, the thickness of the surface layer, wherein the PFD is above the light compensation point (photic zone) is reduced to less than 0.1 cm. For reaching a high quantum yield it is essential that the cells are circulating with a high frequency between the photic zone and the non-avoidable aphotic zone. This way, the mean duration of the aphotic phases and phases of exposition to powered light is reduced and the high PFD can be utilized efficiently by all cells. If the dark phases are very short (millisecond range), they can be utilized for the carbon reduction by using the chemi-osmotic potential that can be stored by the thylakoids in darkness for very short time. In addition, the continuous interruption of the exposition to powered light prevents or shortens the hold up periods in the electron transport chain which might create oxidative stress (Quiang et al. 1998, Pulz 0 2012) In the few studies reporting on rapid photoautotrophic growth at an extremely high cell density with an increase of CDB >4 g l⁻¹ d⁻¹ at a CDB >5 g L⁻¹ (Quiang et al. 1998, Doucha und Livansky 2006), the turbulent flow of the cell suspension was realized in open (non-axenic) systems by gas bubble flow with high velocity or by a high rate of the gravity driven flow rate of the suspension on an inclined flow bed. At exposition to high powered light with a maximum light path of 7 mm, photosynthesis rates of almost 200 g s⁻¹ per m² of the exposed area have been established in a suspension of the cyanobacterium Spirulina platensis for a short period (5 h). Here, the growth limitation by chemical factors (inorganic C and further nutrient dissolved elements) was prevented by a suitable medium. In the open systems exhibiting an intensive gas exchange with the outer gaseous atmosphere it is possible to prevent the enrichment of O₂ in the cell suspension; however, a strong wasting of water by evaporation has to be compensated and the major part of the CO₂ introduced is loosed by the gas exchange with the atmosphere. The substitution of the consumed inorganic C (Cᵢ) in the cell suspension was carried out by delivery of CO₂ from the gaseous phase across the agitated gas-liquid interface (Quiang et al. 1998) or by dissolution of compressed CO₂ (Doucha und Livansky 2006).

Using closed research photobioreactors that are applicable for the axenic culture of PMO at a high cell density (Tsoglin et al., 1995), somewhat lower rates of photosynthesis referred to the exposed area were achieved. In these cases turbation and gas mass transfer was caused by means of a rotor or by shaking. Here however, it happened that the O₂ partial pressure in the closed reaction chamber increased strongly and finally reached growth-inhibiting values.

Besides the already mentioned mass transfer through the turbulent gas-liquid-interface or the dissolution of compressed carbon dioxide, the introduction of carbonized water, the delivery of HCO₃⁻ ions from a cell-free carbon storage medium through a dialysis membrane (DE 2013/102011055481) or the delivery of CO₂ by diffusion across a gas-permeable hydrophobic films or membranes (e.g. US 2004/6,815,204 B2, US 2009/0305389 A1, US 2009/0130704 A1, DE 2009/102008029) are known methods of feeding Cᵢ. A research cultivation system for PMO has been described in DE2009/10 2008 029. According to this method a bag produced from a thin translucent polyethylene film is filled with a concentrated KHCO₃ containing buffer solution and introduced into the shaken culture vessel. Here the utilized Cᵢ is substituted by diffusion of CO₂ across the translucent thin polyethylene film. The growth rates reached with this device were markedly higher than those obtained at shaking the vessels at the air atmosphere. However, this culture system is not suitable for reaching extremely high cell densities (CDB >5 g l⁻¹), since the CO₂ permeability of the applied (translucent) polyethylene films is not high enough for this purpose.

### SUMMARY

The invention applies to a research-photobioreactor for the growth at extremely high cell densities in axenic cultures of cyanobacteria and microalgae exposed to a high light intensity. A hydrophobic gas-permeable first membrane 3 situated at the bottom of the reaction chamber 2 serves for the entry of CO₂ into the cell suspension. The first membrane separates the CO₂ containing gaseous phase in a basis chamber 1 from the cell suspension that covers the membrane. It has a permeability coefficient for the gases of air which many times surpasses the physically feasible rate of absorption of these gases in water. The turbulent flow within the cell suspension required for a high quantum yield is obtained by shaking. A high shearing rate hereby realized increases the CO₂ absorption rate in the liquid phase close to the membrane, thus avoiding C-limitation even at an extremely high volume-based rate of photosynthesis. A porous hydrophobic gas-permeable second membrane 4 separates the reaction chamber 5 from at least one gas exchange chamber 6. The gas exchange chamber is connected with the outer gas atmosphere by means of at least one diffusion limiting channel 7 with a width above 0.01 cm.

The common gas diffusion resistance of all channels 7 is a manifold of the gas diffusion resistance of the second gas-permeable membrane. The surface area A of the reaction chamber A exposed to light as well as the length L and the cross sectional area Q of all channels are chosen such that the geometric factor F=A/Σ(QₙLₙ⁻¹) has a value between 20 and 1000 cm. The gas volume flux through a channel can be determined as measure for the actual photosynthetic turnover. The research-photobioreactor or the reaction chamber may be produced as disposable good. Using the research-photobioreactor according to the invention at a photon flux density of 850 μmol m⁻² s⁻¹, a volume-based rate of the increase in the dehydrated biomass amounting to more than 10 g l⁻¹ d⁻¹ was obtained corresponding to an increase of the dehydrated biomass amounting to more than 40 g m⁻² d⁻¹, if referred to the area exposed to light.

### DETAILED DESCRIPTION

The duty of the invention consists in providing a closed research PBR that is suitable for the axenic culture of PMO with little loss of water and CO₂ and by means of which a daily increase of the dehydrated biomass (DB) of more than 50% is possible at a CDB of more than 10 g l⁻¹ preventing the rise of the O₂ concentration in the cell suspension to growth-limiting values. This duty is achieved by providing a research PBR according to claim 1. The sub-claims relate to favorable embodiments of the research PBR according to the invention.

According to the invention the research-PBR has the following characteristics (FIG. 1):
a closable basis chamber 1 fixed on a horizontal shaker, wherein a CO₂ concentration above one percent (v/v) can be maintained,
at least one reaction chamber 2 for inclusion of the cell suspension that is applied on top of the basis chamber, the inner volume of the reaction chamber being in gaseous exchange with that of the basis chamber exclusively across a planar, micro-porous and hydrophobic gas-permeable first membrane 3, the critical pressure for water infiltration of this membrane amounting to more than 100 kPa, the permeability coefficient to air of this membrane amounting to more than 10 mm³ cm⁻² s⁻¹ kPa⁻¹, and the area of this membrane amounting to more than 30% of the basal area of the reaction chamber,
a porous hydrophobic gas-permeable second membrane separating the reaction chamber 5 from a gas exchange chamber 6 having a critical pressure for water infiltration amounting to more than 100 kPa and a permeability coefficient to air amounting to more than 10 mm³ cm⁻² s⁻¹ kPa⁻¹ at atmospheric pressure,
at least one channel 7 with a width of at least 0.01 cm connecting the gas exchange chamber with the outer atmosphere, the common gas diffusion resistance of all channels surpassing several times the gas diffusion resistance of the gas permeable second membrane, and
the length Lₙ and the cross sectional area Qₙ of the channel(s) are adjusted to the area A of the reaction chamber exposed to light in such a manner that the factor F=A/Σ(QₙLₙ⁻¹) amounts to a value between 20 and 1000 cm.
   A fundamental characteristic of the research-PBR according to the invention consists in the arrangement of a gas exchange chamber in the gas exchange path between the reaction chamber and the atmosphere. The reaction chamber that can be partly filled with the cell suspension is separated by at least one hydrophobic porous gas-permeable second membrane from the gas exchange chamber. Due to the high gas permeability of the second membrane, the gas exchange chamber has approximately the same composition of the gas atmosphere as the reaction chamber. The gas exchange chamber is in connection with the atmosphere through a macroscopic channel with a width of a least 0.01 cm that controls gas diffusion. The channel/s enable/s gas pressure equilibration and control/s the diffusive gaseous exchange between the reaction chamber and the outer gas atmosphere. The diffusive gas exchange between the reaction chamber and the outer atmosphere is controlled by the channel/s, since the common gas diffusion resistance of the channel/s is surpassing the diffusion resistance of the second membrane several times.

Both the undesired diffusive losses of CO₂ and water vapor and the increase of the O₂ partial pressure in the reaction chamber mediated by the photosynthetic reaction depend on the channel length(s) Lₙ and the channel cross-sectional area(s) Qₙ. According to the invention the gas diffusion resistance between the PBR and the atmosphere is controlled by the choice of geometric channel dimensions that together define a resistance factor F. F is an expression of the common resistance to diffusive gas exchange between the atmosphere and the reaction chamber that is referred to the exposed surface area. F is defined by the equation mentioned above. According to the invention, the channel parameters Lₙ and Qₙ are adapted to the exposed surface A area to obtain a value of F between 20 and 1000 cm.

Even at the mentioned upper limit of the F value, the resistance of the channel(s) for the viscous flow of air is so small that the pressure difference between the reaction chamber and the atmosphere is negligible. While the advantage of low F values consists in a rapid diffusion exchange of O₂ and N₂ between the outer atmosphere and reaction chamber, higher F values are favorable by stronger limitation of the water vapor and CO₂ losses to the atmosphere. Research PBR with channel dimension according to a F value close to the mentioned upper limit (1000 cm) cannot prevent a strong increase in the O₂ partial pressure in the reaction chamber at extremely high rates of photoautotrophic DB production (>20 g m⁻² d⁻¹). However, water vapor losses are negligible at such high F values. More technically significant undesired loosing of water vapor and CO₂ is expectable, when F falls below the mentioned lower limit (20 cm). These relationships are exemplified in more detail by the execution example 1.

The research PBR according to the invention can be produced in such a way that the F value is adjustable. For example, when multiple channels are present, this is possible by closing/opening of a part of the channels. When the channel is built as a slit, the F value can be altered by partial closure of the slit.

To obtain a high rate of the CO₂ entry into the suspension at a given CO₂ concentration in the basis chamber, it is favorable, when the first membrane has an area as large as possible and is fixed to the basis of the reaction chamber in such a way that the shearing rate in the liquid phase close to the membrane is as high as possible. To reach a sufficiently high rate of the CO₂ entry into the cell suspension it is essential that the permeable membrane area comprises at least 30 percent of the reaction chambers basis area. Further it is favorable, when the permeable membrane area is located above, not below, the perforations in the basal board of the reaction chamber. An arrangement of the membrane on a gas-permeable porous support layer above the perforated basic board of the reaction chamber is favorable. In this respect, any solid support layer owning high gas permeability in vertical and horizontal direction is designated as gas-permeable support layer. When such a support layer is applied, it is possible to ensure that the gas-saturated area of the first membrane reaches more than 90 percent of the basic area of the reaction chamber, even when the cross sectional area of the perforations is relatively small. This way the area of the first membrane, which is saturated with the gaseous phase of the basis chamber, can reach the value of the reaction chamber surface exposed to the light.

The basis chamber can for instance contain CO₂ with a partial pressure above 1 kPa in it's predominantly gas (air) filled volume. The saturation of the atmosphere in the basis chamber can be achieved by filling some liquid water therein. By means of a reducing valve and a narrow gas inlet, the volume in the basis chamber can be connected to a source of pure CO₂. The set pressure of the reducing valve can be adjusted to a value according to the atmosphere pressure. By means of the reduction valve it is ensured that the partial pressure of CO₂ in the reaction chamber is maintained in the range of the set value even at rapid dissolution of CO₂ in the liquid phase of the reaction chamber.

The same result can also be achieved without gas volume flux from outside, when the CO₂ partial pressure within the basis chamber is maintained by a weakly alkaline buffer solution containing HCO₃⁻ in high concentration. Such buffer solutions are known since a long time as source for providing CO₂ to PMO in experimental vessels (Warburg O und Rippel G 1960). When for instance a buffer solution consisting of 3 M KHCO₃ und 3 M K₂CO₃ is situated in the basis chamber, one can maintain a CO₂ concentration between 1 and 10% (vol/vol) (partial pressure 1-10 kPa) in its gaseous phase for several days even at a high rate of photosynthesis. At the highest measured value of the C-turnover referred to the area exposed to light (Quiang et al 1998) an amount of CO₂ close to 60 mmol per day would be detracted from the basis chamber of a research PBR with an exposed surface area of 1 dm². A manifold of this amount can be delivered by a volume of 300 ml of the mentioned 3 M buffer solution located in the basis chamber without a significant increase in the buffer pH or a significant reduction of the CO₂ partial pressure. The carbon stored in the mentioned buffer can be more completely utilized when the pH value of the buffer is fixed by adding an acid by means of a suitable control unit.

When a weakly alkaline milieu (pH 8.5-10.5) is maintained within the cell suspension by the metabolism of the cells or by a suitable buffer, this is favorable for the growth of numerous PMO. In this case, the major part of Cᵢ dissolved in the cell suspension is existent in the form of HCO₃⁻. This way a strong gradient of the CO₂ concentration in the liquid close to the membrane can be maintained even at a low rate of the actual photosynthetic CO₂ consumption.

A favorable characteristic of the research-PBR according to the invention consists in the fact, that the metabolic O₂ production is the sole significant source of a stationary viscous gas flow out of the reaction chamber into the outer atmosphere. It is, therefore, possible to measure the volume flow out of the gas exchange compartment into the atmosphere as measure of the actual photosynthetic turnover rate in a research PBR with the manifested characteristics. If for instance, a capillary tube is connected to a channel, the volume flux through this tube can be determined as measure of the actual photosynthesis rate. A simple possibility for such measurement is explained in the execution example 2.

When concentrated HCO₃⁻ containing buffer solutions with a concentration of the alkali ions above 3 M are applied to maintain the CO₂ concentration in the basis chamber, the partial pressure of water vapor above this buffer is markedly lower than that in equilibrium with the cell suspension at the same temperature. In the isothermal case a transport of water vapor from the cell suspension across the first membrane into the buffer solution is not avoidable. At 30° C. this would result in a water loss amounting to some percent of the suspension volume per day. For an accurate measuring of the biomass production, it is therefore, essential to measure the water loss besides the measurement of the CDB. The volume change by isothermal distillation of water from the cell suspension to the buffer solution can be avoided or strongly diminished by maintaining a difference between the temperature of the buffer solution in the basis chamber and the temperature of the cell suspension in the reaction chamber. By thermal approaching the absolute partial pressure of water vapor in the basis chamber to that in the cell suspension, it is possible to prevent or reduce the osmotically mediated process of water vapor distillation across the first membrane. The required temperature difference can be obtained by means of an electric heater. A further possibility for creation of such a temperature difference consists in fortification of the cooling facility used for deduction of the radiant heat from the reaction chamber. This will be explained in the execution example 2.

Due to the limiting role of gas absorption at the gas-liquid-interface it is favorable, when the first membrane shares a large portion of the basis area of the reaction chamber. However, one can abstain from a large area of the second membrane, if the latter is located in a compartment above the suspension, where it is not wetted by the suspension. It is required that its gas permeability is high, as is the case for the already mentioned membranes of the type Celgard or Treopore. A small area of the second membrane is favorable in order to reduce a shading of the cell suspension, since the porous membranes are light-reflecting. Only the gas-permeability of the second membrane is crucial for the mass exchange across this membrane, when the membrane is not wetted with a liquid. If the second membrane were relatively small and covered by liquid film, a strong hindrance of the gaseous exchange would be achieved due to the low rate of gas absorption and de-sorption at a gas-liquid-interface. Although a continuous wetting of a hydrophobic membrane by splash contact with the suspension was not expected, the experience showed that it is favorable at high cell densities; to arrange the second membrane in a compartment of the reaction chamber, wherein it is protected from splashing with the cell suspension (FIGS. 3 and 4).

The research-PBR or its reaction chamber can be produced in disposable form using porous and hydrophobic membranes with the characteristics of the invention, for example the commercially available membranes described above, and preferably translucent plastics like polystyrene, or polycarbonate. A favorable embodiment of the research-PBR consists in the combination of a relatively large number of exchangeable reaction chambers with one large basis chamber. For the study of parallel batches this has the advantage, that all investigated cell suspension can be exposed to the same photon flux density, the same intensity of turbulent flow and the same conditions for CO₂ entry. In this case it is favorable, when the basis chamber has no direct fluidic connection with the atmosphere.

### BRIEF DESCRIPTION OF THE DRAWINGS

Figures (schematic, not drawn to scale)

FIG. 1: A cross section through a research PBR with essential characteristics of the invention.

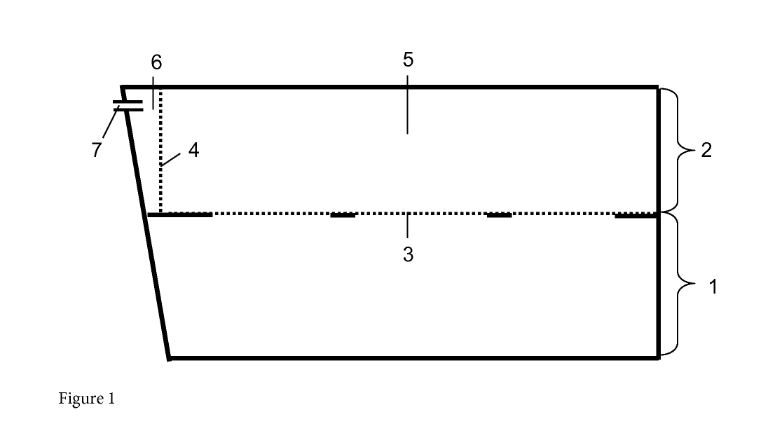

FIG. 2: A cross section through a research PBR with a suspension of PMO, whereat the CO₂ concentration in the gaseous phase of the basis chamber is maintained by introduction of pure CO₂ through a reducing valve,

FIG. 3: A cross section through a research PBR with a suspension of the PMO, wherein the CO₂ concentration in the gaseous phase of the basis chamber is maintained by a buffer with a high concentration of HCO₃⁻ ions. The current rate of photosynthesis can be measured with a soap-foam flowmeter. The second membrane is located within a compartment of the reaction chamber, wherein it is protected from wetting with the cell suspension at shaking.

FIG. 4: A cross section through two research PBRs with a reaction chambers as in FIG. 3. Filling devices and silicon stopper are not drawn. The surface A exposed to the light amounts to 25 cm² in both PBRs. The channel of PBR shown at the left side has a length L of 2 mm and a cross sectional area Q of 6.3 mm² (F=79.4 cm), whereas the channel of the PBR shown at the right side has a cross sectional area Q of only 3.14 mm² and a length L of 10 mm (F=794 cm).

FIG. 5: A cross section of an experimental device for comparison of the membrane-mediated rate of CO₂ absorption with the rate of CO₂ absorption through the free gas-liquid interface at the border between the liquid phase and the gas space within the chamber. Filling device and silicon stopper are not drawn. In the reaction chamber on the left side the CO₂ entry into the liquid within the reaction chamber happens by dissolution from the gas-saturated membrane as in the research PBR according to the invention. In the reaction chamber shown at the right side the CO₂ entry into the liquid phase of the reaction chamber happens according to the principle described by Warburg and Krippahl (1960) across the agitated gas-liquid interface.

### DETAILED DESCRIPTION OF THE PREFERRED EMBODIMENT

### Execution Example 1

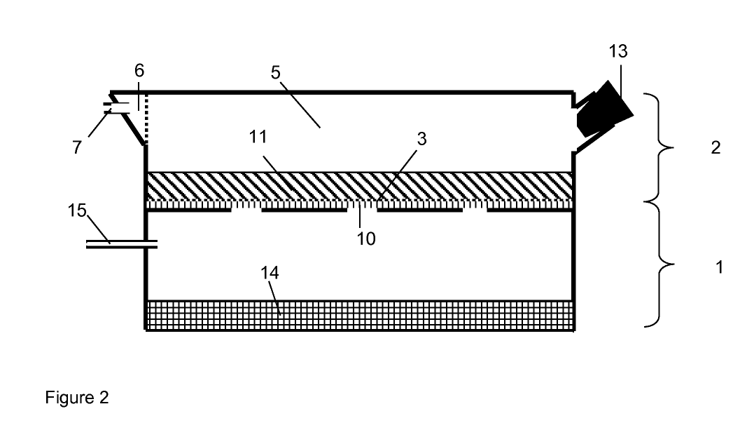

The limits of the geometric resistance factor of a research PBR according to the invention, F=A/Σ(QₙLₙ⁻¹), which is defined by the exposed surface area of the reaction chamber A and the channel parameters cross sectional area Q and L will be explained for the PBR schematically shown in FIG. 2. The surface A of this PBR amounts to 1 dm². The PBR has two channels, each with a cross sectional area of 0.2 cm². The first channel having a length L of 0.1 cm can be closed; the second one is permanently open has a length L of 1 cm. The resulting permeability G of both channels for the diffusion exchange of O₂ at atmospheric pressure can be expressed as G=D (Q₁L₁⁻¹+Q₂L⁻¹), wherein D symbolizes the diffusion coefficient of the air for O₂. Inserting of the numeric values for Q, L, and D into this equation, a value for G close to 0.44 cm s⁻¹ is achieved. For the research-PBR described above, the geometric resistance factor that is related to the exposed surface area F=A/G=A/Σ(QₙLₙ⁻¹ has the value 45.5 cm. The resistance for a viscous air flow within the longer of the two channels (L=1 cm) can be estimated by the Hagen/Poisieulle law under consideration of the viscosity of air (17.1 μPa s at 20° C.) and is close to 0.684 mPa s cm⁻¹ assuming a circular cross section win an area of 0.2 cm².

According to the duty of the invention the research-PBR should be suitable for high rates of photosynthetic turnover. Hence the channel dimensions are chosen in such a way that an increase in DB of 200 d⁻¹ per m² of the PBR surface exposed to the light, which slightly surpasses the highest value so far measured, does not induce a strong increase of the O₂ concentration in the reaction chamber. The portion of carbon in the DB is similar to that in carbohydrate (CH₂O). Hence at normal temperature (20-30° C.) the rate of O₂ formation coupled to an increase in DB of 200 g m⁻² d⁻¹ is about 6.67 mol d⁻¹, which corresponds to 167 l m⁻² d⁻¹ or 0.000193 cm³ cm⁻² s⁻¹. In a research PBR with an exposed surface of 1 dm⁻² the rate of oxygen formation by one chamber would be 0.193 cm³ s⁻¹ or 1.67 l per day. At the mentioned extremely high rate of photosynthesis the basis chamber should include a gas volume of at least 5 l in order to dilute the O₂ volume formed at one day efficiently in its space. At the channel dimensions according to the invention the increase in the O₂ concentration can be avoided by diffusive exchange of atmospheric N₂ with the O₂ formed in the reaction chamber. The stationary concentration difference □C between the space within the reaction chamber and the atmosphere is connected with the geometric resistance factor F, the diffusion coefficient D of O₂ in air, and the rate of O₂ formation per unit of the area exposed to light by the equation

ΔC=JFD⁻¹

When a value for D representative for physiologic temperatures at atmospheric pressure (0.2 cm² s⁻¹) as well as values for J (0.000193 cm³ cm⁻² s⁻¹) and the geometry factor mentioned above (45.5 cm) is inserted into this equation, this results in a concentration difference ΔC amounting to 0.044 in the expression as molar ratio. This correlates with an enhancement of the O₂ concentration in the reaction space by 4.4% (vol/vol) and a stationary O₂ concentration within the reaction chamber of about 23.5%, when PBR is shaken in air. Hence, at the chosen channel dimensions (L=0.1 cm, Q₁=0.2 cm², Q₂=0.1 cm, L2=1 cm) and an area of the PBR exposed to the light of 1 dm², a physiologically relevant enrichment of oxygen within the reaction chamber does not occur even at an extremely high photoautotrophic turnover referred to the exposed surface.

The gas flow within the first (shorter) channel is larger than that in the second longer one. When the gas volume flow out of the PBR is 0.0193 cm³ s⁻¹ (=100 cm²×0.000193 cm³ cm⁻² s⁻¹) as explained above, in the first channel (0.1 cm) it amounts to 0.0175 cm³ s⁻¹ and in the second one (1 cm) it amounts to 0.00175 cm³ s⁻¹. The velocity of viscous gas flow in the shorter channel would approach a value of 0.035 cm s⁻¹, which is much below the velocity of O₂ diffusion in this channel. The latter can be regarded as the permeability coefficient D/L of a 0.1 cm thick air layer for O₂, which amounts to a value of about 2 cm s⁻¹. Since the permeability coefficient surpasses the flow velocity about 57 times, the effect of the viscous gas flow on the diffusion exchange of N₂ against O₂ can be neglected. Due to the low value for the viscous flow resistance obtained in the longer channel (L=1, circular cross section) a nearly ideal pressure equilibration between the exchange space an the atmosphere would be achieved at the above mentioned extremely high value of the O₂ formation rate in the reaction chamber (0.0192 cm³ s⁻¹) even when the shorter channel would be closed. The pressure drop along the longer channel at this volume flux would be as small a about 0.32 mPa.

In a research PBR with an exposed surface amounting to 1 dm² having a single channel with a cross sectional area Q of 0.05 cm² and a length of 1 cm the factor F would be 2000 cm. At the extremely high rate of O₂ formation per chamber (0.0193 cm³ s⁻¹) the flow velocity in the channel would approach to 0.386 cm s⁻¹. In this case the velocity of viscous gas flow would surpass the permeability coefficient of a layer of unstirred air with the thickness 1 cm for the exchange of N₂ against O₂ (0.2 cm s⁻¹). Hence, N₂ initially present within the air volume in the reaction chamber would be completely driven out of the reaction chamber by the viscous gas volume flow. Hence, a PBR with a resistance factor of 2000 cm can prevent oxidative stress only at rates of photosynthesis referred to exposed area that are fairly below the maximum so far measured.

Due to the limited saturating value of the water vapor concentration in air, the difference in the water vapor concentration between the inner space of the reaction chamber and the atmosphere cannot reach more than 30 mg l⁻¹ or 4.2 percent (vol/vol) at a temperature of 30° C. The possible loss of water vapor also depends on the geometry factor F and is maximum at the lower limit for this factor according to the invention (F=20 cm). At this value of F and a water vapor concentration difference of 30 mg l⁻¹ the calculated possible loss of water vapor amounts to about 1.2 g d⁻¹, which represents 2.4% of the suspension volume at a thickness of the suspension layer of 5 mm.

Even lower values are obtained for the loss of CO₂ through the channel(s), since the CO₂ partial pressure in the basis chamber can be adjusted to a value that is fairly below that of water vapor. Further, when a weakly alkaline buffered medium is applied, wherein the absorbed CO₂ is transformed to HCO₃⁻ before its consumption by the PMO, the CO₂ partial pressure above the cell suspension remains very small, independent of the photosynthesis rate. In this case, the CO₂ loss to the atmosphere is not technically relevant.

### Execution Example 2

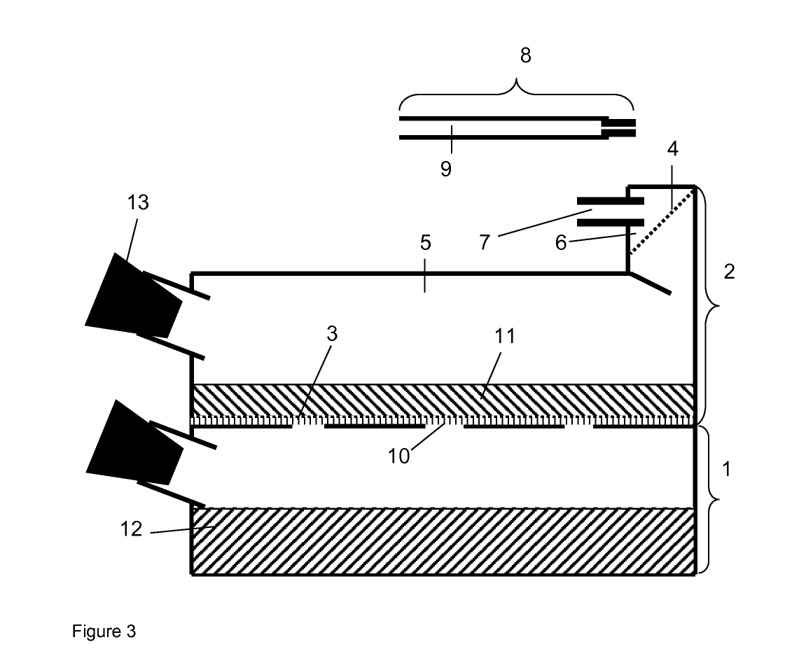

Research PBRs of the type shown in FIG. 3 were applied for the culture of the cyanobacterium Synechococcus sp. PCC 7002. 50 ml-culture vessels made from polystyrene were used for building the basis chamber as well as the reaction chamber.

The size of the surface area A exposed to the light was 25 cm², the cross sectional area Q of the channel amounted to 0.063 cm², and a channel length L of 0.2 cm was chosen. A polypropylene network served as gas permeable support layer; A 40 μm thick Treopore membrane (Type PDA) was applied as gas permeable membrane. The tight seal of the membrane on the basic board of the reaction chamber, the connection of both chamber and further modification of the culture vessels were carried out by gluing with transparent silicone. The sterilization of the reaction chamber was carried out by introduction of an 11% aqueous H₂O₂ solution (20 ml) into the vessel. After short panning and longer incubation (about 6 h) the major part of the oxidant solution was poured off and, after closure of the vessel with a silicon stopper, the residual liquid (1-2 ml) was allowed to evaporate across the gas-permeable membranes.

A culture medium was applied, which contained al macronutrient in a concentration that was sufficient for DB formation of 30 g l⁻¹ referred to the suspension volume. The medium which was found to be suitable for the mentioned organism contained 150 mM NO₃⁻, 100 mM K⁺, 10 mM HPO₄⁻, 10 mM SO₄; 10 mM Cl⁻, 10 mM Mg²⁺, 0.5 mM Fe²⁺, 0.5 mM EDTA, 0.5 μM Vitamin B₁₂, 2 mM H₃BO₄, 100 μM Mn²⁺, 10 μM Zn²⁺, 6; MoO₄, 0.2 μM Co²⁺ and 0.05 μM Cu²⁺. It was buffered with 100 mM NaHCO₃. The culture medium was prepared by mixing at room temperature under a sterile flow box by adding one volume of a separately autoclaved 1M CaCl₂ solution to 100 volumes of the autoclaved common solution of the other constituents. The reaction chamber contained 20 ml of this culture medium and the basis chamber was filled with 30 ml of a buffer obtained by mixing of 1 volume of a 3 M K₂CO₃ with 3 volumes of a 3 M KHCO₃ solution.

The research PBR was fixed on 5 mm thick black-painted aluminum board on the shaking desk. There were holes within the board, into which the research PBRs could be inserted. The shaking desk was exposed to the light of two green house lamps (each 400 W), the light of which was filtered by 7 mm thick glass plate. The glass plate and the shaking desks were cooled by an air flow from aside. Using a board with adjustable height whereon the shaking desk was fixed the PFD could be adjusted to a value between 200 and 1200 μmol m⁻² s⁻¹. This device was located in a room with low relative humidity (<40%) of the air and an air temperature of 28° C. An efficient cooling of the exposed PBRs was ensured by the air flow. At high values of the PFD (>500 μmol m⁻² s⁻¹) the temperature of the reaction vessels was only slightly higher (less than 2° C.) than that of the air, while the temperature of the aluminum plate on which the basis boards of the PBRs were fixed, was higher (3 to 4° C. above the air temperature). This temperature difference between the basis chamber and the reaction chamber was sufficient to prevent a significant loss of water to the reaction chamber by osmotically mediated vapor distillation.

The PBRs were applied in the repeated batch mode, while they were shaken with 10 or 20 ml of the culture medium on a circular shaker adjustable at 200 to 400 rpm. The daily increase of the CDM was determined after strong dilution (1/100 to 1/20) with a spectral photometer by measuring the light attenuation at a wavelength of 750 nm using a calibration curve. Sampling during a growth cycle was carried after by removal of a small volume of the suspension (0.3 ml) through a closable and heat-sterilizable port, while the sampling at the start and the end of each cycle was carried out before dilution of the suspension, which was carried out under the sterile flow box. Inoculation was carried out with an initial CDB of about 0.3 g l⁻¹. Already after one day there was stated a CDB of 2 to 3 g l⁻¹. The increase of the CDB on the following days depended on the PFD in a nearly linear manner in the PFD range between 400 and 1200 μmol m⁻² s⁻¹.

To realize the repeated batch culture regime the cell suspension was diluted with the fresh nutrient solution in two day cycles. The suitable dilution rate was calculated and defined after measuring the CDB to a value ensuring that the CDB after dilution reached approximately 2 g l⁻¹ as the initial value of the next cycle.

At a PFD of 850 μmol m⁻² s⁻¹ the CDB increased from about 2 g l⁻¹ to a value amounting to 25 to 27 g l⁻¹ within 48 h. This corresponds to an absolute value of the daily increase in the DB of more than 0.2 g per reaction chamber or of more than 40 g per m² of the area exposed to light. The increase in the DB per reaction chamber was largely independent of the thickness of the cell suspension layer (4 or 8 mm), from which it may concluded that PFD was the limiting factor. Probably even higher photosynthetic turnover rates could be obtained at higher values of the PFD. At a volume of 20 ml of the cell suspension (thickness of the unshaken suspension 8 mm) the DB reached 12 to 15 g l⁻¹ after the second day. At a suspension layer thickness of 4 mm the daily increase of the DB, referred to volume, was 12 to 13 g l⁻¹.

Using the measuring device illustrated in FIG. 3 the gas volume flux out of the reaction chamber into the atmosphere, which occurs at approximately constant values of the temperature and the pressure in the reaction chamber exclusively by photosynthetic gas formation, could be recorded. For this purpose a 40 μl capillary containing colored tenside foam segment was connected to the channel, whereat the shaking was interrupted for about one second. Subsequently the time required for the flow of the foam segment through a 20 mm³ segment of the capillary was measured. When the photosynthetic gas flow was studied at different light intensities a tight correlation between the measure flow rate and the increase in the DB determined at the day before measurement was found. At a high PFD measured volume flow rates reached values of 1 to 2 mm³ s⁻¹. No measurable gas volume flow happened after closing the light. The applied soap foam flow-meter does not induce a significant increase in the gas pressure within the reaction chamber and is, therefore, suitable to measure the current rate of the O₂ release. Assuming C assimilation as carbohydrate (CH₂O), a theoretical value for the increase in DB was calculated from the volume flux obtained with the flow-meter. The value measured at the day before the flow measurement (first day) amounted to about 80% of the calculated value obtained this way.

### Execution Example 3

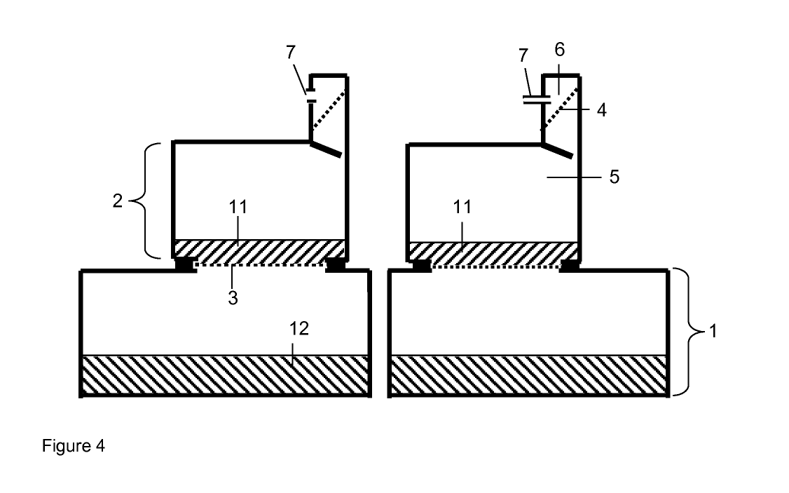

Two research PBRs according to FIG. 4 were used to demonstrate the influence of the channel dimensions on the photosynthetic turnover rate of Synechococcus sp. PCC 7002

The suspension volume amounted to 20 ml (layer thickness 8 mm). The channel of the research PBR shown at the right side was more narrow and longer than at the one shown in the PBR shown at the left side. The duration of the culture (dilution) cycle was two days. In the PBR shown at the right side the resistance factor of diffusive gaseous exchange per area exposed to the light was smaller (F=79.4 cm) than in that shown at the left side (F=794 cm). Here the increase in DB at the second day was slightly larger than at first day and amounted to more than 40 g m⁻² d⁻¹. In the chamber variant with the larger resistance of diffusive gaseous exchange the increase in the DB was not different from that in the parallel variant at the first culture day. At the second culture day, however, it was markedly smaller (25 to 35 g m⁻² d⁻¹) than in the parallel variant. The result was found without exception in 5 following cycles. When the F value of the chamber variant shown left was reduced from 794 cm to 79.4 cm by shortening the channel from 1 cm to 0.1 cm, in this variant the increase in DB during the second day also surpassed the value of the first day and a significant difference between both variants could not be stated anymore. It can be deduced from the equation ΔC=F J D⁻¹ (see first execution example) that the O₂ concentration in the reaction chamber can rise above the value of the outer atmosphere only a little (by 1.5% vol/vol), when F=79.4 at a daily increase in dry weight referred to the exposed area of 40 g per m² (J=38.6.×10⁻⁴ cm³ cm⁻² s⁻¹). The equation also shows that steady state value for the O₂ concentration must be markedly higher (ca. 35% vol/vol) than that of air at the same rate of photosynthetic turnover at an F value of 794 cm. The results show that the O₂ concentration in the chamber with the larger F value reached a growth-inhibiting value only at the second day. This can be explained by the fact that at each dilution of the cell suspension there was an exchange of the relatively large gas volume in both chambers of the research PBR with the atmosphere.

### Execution Example 4

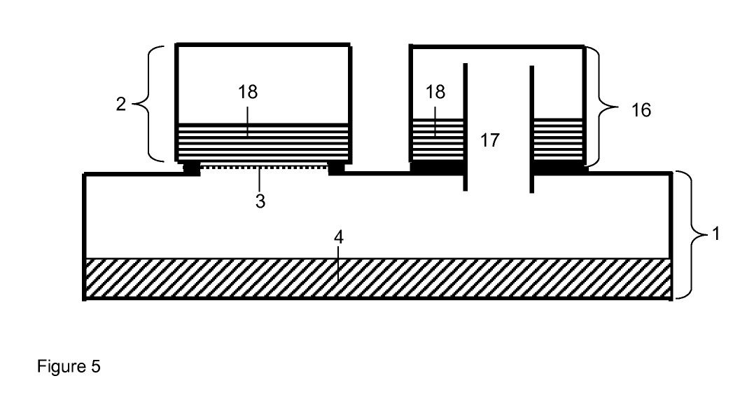

The experimental device shown in FIG. 5 was applied to compare the rates of CO₂ absorption into a weakly alkaline buffer from the surface of the first gas-permeable membrane (Treopore/PDA of the Treofan Company) with those obtained for the CO₂ absorption from the agitated free air/liquid interface. As distinguished from the normal application of the research PBR, the reaction chambers were completely closed against the outer atmosphere. A common basis chamber was produced with the help of 550 ml polystyrene culture vessel. It was filled with 200 ml of a buffer produced by mixing of four volumes of a 3 M KHCO₃ solution with one volume of a 3 M KHCO₃ solution. The CO₂ partial pressure in the gaseous phase equilibrated with this buffer is declared as 3.2 kPa (3.2%, vol/vol). The two reaction chambers fixed on top of the basis chamber were produced from 50 ml-polystyrene culture vessels. There was filled 19 ml deionized water into each of these chambers. Then the experimental design was shaken for one hour to saturate the liquid and gas volumes with CO₂. Finally one ml of a solution containing 0.1M NaHCO₃ and 0.1 M K₂CO₃ as well as 10 μl of 1% solution of phenolphthalein in ethanol were introduced into both reaction chambers. The reaction chambers were closed and the time required for the bleaching of the indicator was determined. This time is consistent with the time required for the absorption of 0.1 mmol CO₂, since at bleaching of phenolphthalein (pH 8.5 to 9) the concentration of CO₃²⁻ions has reached zero. The table shows the mean rates of CO₂ entry into the buffer solution at the shaking frequencies applied (100, 200 and 400 rpm). The membrane-mediated rate of CO₂ absorption was markedly higher than the rate of CO₂ absorption through the free gas-liquid interface. The result is even more striking if it is considered the values of CO₂ entry through the free gas-liquid interface were referred to the non-agitated liquid surface. The latter are much higher than those expected for the much larger (but not measurable) area of the agitated air-liquid interface. The CO₂ absorption was increased only slightly by increasing the shaking frequency from 200 rpm to 400 rpm, whereas an increase from 100 to 200 rpm had a strong positive effect.

TABLE
CO₂-Absorption in an alkaline buffer solution
shaken in the reaction chamber

| | A: CO₂-absorption across | B: CO₂-absorption across | |
|---|---|---|---|
| shaking | the first membrane | the gas-liquid interface | |
| frequency | (FIG. 5 left scheme | FIG. 5 right scheme | |
| (rpm) | [μmol cm⁻² hr⁻¹] | [μmol cm⁻² hr⁻¹]* | A/B |
| 100 | 19.0 | 15.6 | 1.22 |
| 100 | 18.6 | 16.1 | 1.15 |
| 200 | 32.2 | 20.7 | 1.56 |
| 200 | 34.0 | 21.0 | 1.61 |
| 200 | 32.9 | 21.0 | 1.57 |
| 400 | 34.3 | 21.4 | 1.60 |
| 400 | 38.2 | 24.1 | 1.58 |

*referred to the surface area in non-shaken vessel

At the higher frequencies of shaking the mean rate of CO₂ absorption from the membrane area amounted to 34.2 μmol cm² s⁻¹. This would enable a daily rate of photosynthesis according to the consumption of 8.2 mol CO₂ per m² d⁻¹ or the formation of 246 g CH₂O m⁻² d⁻¹, which surpasses the highest rates of photosynthesis per unit area exposed to light so far known. Even higher rates of CO₂ absorption were obtained, when a 1 M KOH was shaken in the experimental device. This was expected, since the reaction of the dissolved CO₂ to bicarbonate and carbonate is extremely rapid at the high concentration of OH⁻ ions in this solution and can prevent the saturation of the liquid at the interface with CO₂ (Edsall 1969)

### LIST OF REFERENCE NUMERALS

1 basis chamber
2 reaction chamber,
3 first membrane,
4 second membrane,
5 reaction volume,
6 gas exchange chamber,
7 channel,
8 soap-foam flowmeter
9 calibrated capillary tube
10 gas permeable support layer
11 cell suspension
12 buffer solution composed of 3 M KHCO₃ and 3 M K₂CO₃
13 silicone stopper
14 water saturated matrix
15 CO₂-inlet
16 reference reaction chamber
17 tubular connection
18 20 ml of 25 mM K₂CO₃ and 25 mM KHCO₃ stained with phenolphthalein

### PATENTS CONSIDERED

DE 2013/102011055481, US 2004/6,815,204 B2, US 2009/0305389 A1, US 2009/0130704 A1, DE 2009/102008029, DE2009/10 2008 029

### REFERENCES

Doucha, J., & Livansky, K. (2006). Productivity, CO2/O2 exchange and hydraulics in outdoor open high density microalgal (Chlorella sp.) photobioreactors operated in a Middle and Southern European climate. Journal of Applied Phycology, 18(6), 811-826.
Edsall, J. T. (1969). Carbon Dioxide, Carbonic Acid and Bicarbonate Ion: Physical Properties and Kinetics of Interconversion. NASA SP-188, CO2: Chemical, Biochemical, and Physiological Aspects, p. 15.
Pulz, O. (2001). Photobioreactors: production systems for phototrophic microorganisms. Applied Microbiology and Biotechnology, 57(3), 287-293.
Qiang, H., Zarmi, Y., & Richmond, A. (1998). Combined effects of light intensity, light-path and culture density on output rate of Spirulina platensis (Cyanobacteria). European Journal of Phycology, 33(2), 165-171.
Tsoglin, L. N., Gabel, B. V., Falkovich, T. N., & Semenenko, V. E. (1996). Closed photobioreactors for microalgal cultivation. Russian Journal of Plant Physiology, 43(1), 131-136.
Wang, B., Lan, C. Q., & Horsman, M. (2012). Closed photobioreactors for production of microalgal biomasses. Biotechnology Advances, 30(4), 904-912.
Warburg, O., & Krippahl, G. (1960). Weiterentwicklung der manometrischen Methoden (Carbonatgemsiche). Z. Naturforschg., 15b, 364-367.

1. Research photobioreactor for the warranty of the photoautotrophic growth of cyanobacteria and microalgae under axenic conditions at extremely high photoautotrophic activities with small losses of CO₂ and water to the atmosphere, comprising
a horizontally orientated basis chamber 1 that can be closed from the atmosphere and fixed on a shaking desk, wherein a CO₂ concentration above 1% (vol/vol) can be maintained,
at least one horizontally extended reaction chamber 2 for inclusion of the cell suspension that is fixed on the upper board of the basis chamber, the volume of which persists in gaseous exchange with the basis chamber exclusively through a horizontally orientated planar, microporous and hydrophobic first membrane 3, whereat the critical pressure of this membrane for infiltration by water in the air-saturated state surpasses 100 kPa and its permeability for air at atmospheric pressure surpasses 10 mm³ cm⁻² s⁻¹,
a porous hydrophobic gas-permeable second membrane 4 with a critical pressure for infiltration with water in the air-saturated state of more than 100 kPa and a permeability to air at atmospheric pressure of more than 10 mm³ cm⁻² s⁻¹ kPa⁻¹, separating the reaction volume 5 from a gas exchange volume 6, as well as
at least one channel 7 connecting the gas exchange volume with the outer atmosphere having a width of at least 0.01 cm whereat the common gas diffusion resistance of all channels surpasses several times that of the second gas-permeable membrane and the surface area A of the reaction chamber exposed to light, whereat the length Lₙ and the cross sectional area Qₙ of each channel are chosen in such limits, that the geometry factor F=A/Σ(QₙLₙ⁻¹) is a value between 20 and 1000 cm.

2. Research photobioreactor according to claim 1, having a device for measuring the gas volume flow from the gas exchange volume to the atmosphere.

3. Research photobioreactor according to claim 2, whereat the device for measuring the gas volume flow is a soap-foam flow-meter 8.

4. Research photobioreactor according to claim 1, with a gas-permeable support structure 10 which is located between the perforated basic board of the reaction chamber and the first membrane, whereat the gas-permeable area of the first membrane comprises more than 90% of the basis area of the reaction chamber.

5. Research photobioreactor according to claim 1, whereat the second membrane is bordering to a compartment of the reaction volume where it is protected from permanent wetting with the cell suspension.

6. Research photobioreactor according to claim 1, with a device for maintaining a temperature difference between the liquid phase in the basis chamber and the cell suspension that is reducing the condensation of water vapor in the liquid phase of the basis chamber.

7. Research photobioreactor according to claim 1, having several exchangeable reaction chambers that can be fixed to a common basis chamber at one or several closable ports in its cover board.
